(19) United States
(12) Patent Application Publication
(10) Pub. No.: US 2015/0053298 A1
(43) Pub. Date: Feb. 26, 2015

(54) Anti-Kink Tubing

(71) Applicants: Kevin Alan Tussy, Las Vegas, NV (US); Brian Keith Leffler, Martinez, CA (US)

(72) Inventors: Kevin Alan Tussy, Las Vegas, NV (US); Brian Keith Leffler, Martinez, CA (US)

(21) Appl. No.: 14/464,539

(22) Filed: Aug. 20, 2014

Related U.S. Application Data
(60) Provisional application No. 61/867,910, filed on Aug. 20, 2013.

(51) Int. Cl. F16L 11/08; F16L 11/12; A61M 39/08

(52) U.S. Cl. CPC F16L 11/08; A61M 39/08; F16L 11/12; A61M 2039/0009
USPC 138/118; 138/177

(57) ABSTRACT

Anti-kink tubing includes a hose or tube having an inner surface and an outer surface that form an inner enclosed cavity that defines a central axis of the tube. A plurality of anti-kink projections attach to at least one of the inner and outer surfaces. The plurality of anti-kink projections may extend along at least one of the inner and outer surfaces in a direction generally parallel to the central axis of the anti-kink tubing.

### 1. FIELD OF THE INVENTION

The disclosed embodiments relate to tubing or hoses. More specifically, the disclosed embodiments relate to tubing or hoses that reduces the likelihood of kinking.

### 2. RELATED ART

Existing designs for tubing or hoses can sometimes become doubled over, or kinked, such that flow is restricted within the tubing or hose. This is especially problematic in delivering fluids through intravenous (IV) tubing where interrupted or substantially constrained flows can mean that important drugs or other fluids are not delivered as expected. IV tubing is especially prone to kinking as the tubing may get twisted and otherwise kinked as the patient moves around or rolls over in a bed.

One proposed solution is to wrap a spiral metal wire around the tubing such that the metal wire is sufficiently thick and ridged to prevent kinking of the hose. While this proposed solution does reduce the likelihood of a kink, it suffers from numerous drawbacks. One such drawback is that the wire adds significant weight to the tubing, thereby making the tubing difficult to carry, such as if used as a long garden hose. Another disadvantage is that the ridged wire reduces flexibility of the hose thereby reducing its ability to bend and move around a corner or obstacle. A further drawback is that if the hose does kink, it is likely to permanently bend the wire, which in turn permanently established the kink at that location.

Accordingly, there is a need for lightweight tubing that reduces the likelihood of kinking while maintaining flexibility of the tubing.

### SUMMARY

The disclosed embodiments are developed light of the above described problems and some aspects of the invention may include anti-kink tubing that comprises an inner surface, an outer surface, and a plurality of anti-kinking projections attached to at least one of the inner and outer surfaces. The plurality of anti-kinking projections may extend along the at least one of the inner and outer surfaces in an axial direction of the anti-kink tubing.

The anti-kink tubing described above my further have anti-kinking projections that extend along an entire axial length of the tubing. In the anti-kink tubing, the inner surface may be substantially circular in cross section, and the anti-kinking projections may be formed integrally with the outer surface and protrude from the outer surface.

In another example of anti-kink tubing, the anti-kinking projections may be formed on the outer surface, and a profile of the inner surface may substantially conform to that of the outer surface. As a further alternative, the anti-kink tubing may have anti-kinking projections that are formed on the inner surface, and comprise partitions that extend radially through an inside of the tubing.

According to other aspects of the invention, anti-kink intravenous tubing may comprise an inner surface, an outer surface, and a plurality of anti-kinking projections attached to at least one of the inner and outer surfaces. The plurality of anti-kinking projections may extend along the at least one of the inner and outer surfaces in an axial direction of the anti-kink tubing. Accordingly, the plurality of anti-kinking projections may define reinforcing ribs on the at least one of the inner and outer surfaces.

In the anti-kink intravenous tubing the plurality of anti-kinking projections may be disposed on a distal part of the intravenous tubing. The plurality of anti-kinking projections may further be formed in a substantially triangular shape in cross section. Alternatively, the plurality of anti-kinking projections may form a plurality of flutes along the outer surface, or the plurality of anti-kinking projections may be formed in a substantially semi-circular shape in cross section.

According to further aspects of the invention, an anti-kink garden hose may comprise an inner surface, an outer surface, and a plurality of anti-kinking projections attached to at least one of the inner and outer surfaces. The plurality of anti-kinking projections may extend along the at least one of the inner and outer surfaces in an axial direction of the anti-kink hose. A proximal end of the garden hose may be configured to connect with a water spout, and the plurality of anti-kinking projections may be disposed on the proximal end.

In the anti-kink hose, the plurality of anti-kinking projections may be formed on a sleeve, and the sleeve may be configured to fit on the outer surface of the anti-kink garden hose. The anti-kinking projections on the anti-kink hose may define reinforcing ribs on the at least one of the inner and outer surfaces.

Also disclosed is anti-kink tubing comprising a flexible tube having an inner surface and an outer surface that define an inner cavity extending in an axial direction. Also part of the tube are a plurality of anti-kinking projections attached to at least one of the inner and outer surfaces such that the plurality of anti-kink projections extend along the at least one of the inner surface and outer surfaces parallel to the axial direction of the anti-kink tubing.

In another embodiment, a flexible hose is defined by a wall having an inner surface and an outer surface which form an inner cavity that extends from a first hose end to a second hose end to define a center axis generally centered in the inner cavity. The outer surface of the hose forms a plurality of projections extending from the wall to establish the outer surface at different distances from the inner wall based on the circumferential location on the outer surface. The projections form ribs which extend from the first hose end to the second hose end generally parallel to the central axis.

In one configuration the anti-kink intravenous tube further comprises a first connector on the first hose end and a second connector on the second hose end. In one embodiment, the projections are formed in a substantially triangular shape in cross section. In one exemplary configuration the projections form a plurality of flutes along the outer surface.

Also disclosed is an anti-kink tube comprising a tube having a central axis such that the tube has an inner surface and outer surface which define an inner cavity. One or more of anti-kink projections are part of the tube and extend from the inner surface within the inner cavity and are formed as part of or attached to the inner surface. The anti-kink projections extend from the inner surface toward the central axis such that the anti-kink projections run parallel to the central axis of the tube.

In one embodiment, at least one of the one or more anti-kink projections has a first end and a second end, the second end opposing the first end, such that both the first end and the second end contact the inner wall and the anti-kink projection extends through the central axis. In one configuration, the inner surface and the outer surface are equal distance apart around at least a portion of the circumference of the anti-kink tube. In one variation, the anti-kink projections comprise two anti-kink projections which are equally spaced within the cavity and each of the two anti-kink projections extend through the central axis.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1A is an axial view of an anti-kink tubing according to one exemplary embodiment.

FIG. 1B is a perspective view of the anti-kink tubing shown in FIG. 1A.

FIG. 2A is a cross section view of an anti-kink tubing according to an exemplary embodiment.

FIG. 2B is a cross section view of the anti-kink tubing shown in FIG. 2A where the anti-kink tubing is curved.

FIG. 3A is an axial view of an anti-kink tubing according to one exemplary embodiment.

FIG. 3B is a perspective view of the anti-kink tubing shown in FIG. 3A.

FIG. 4A is an axial view of an anti-kink tubing according to one exemplary embodiment.

FIG. 4B is a perspective view of the anti-kink tubing shown in FIG. 4A.

FIG. 5A is an axial view of an anti-kink tubing according to one exemplary embodiment.

FIG. 5B is a perspective view of the anti-kink tubing shown in FIG. 5A.

FIG. 6 is an axial view of an anti-kink tubing according to one exemplary embodiment.

FIG. 7 is an axial view of an anti-kink tubing according to one exemplary embodiment.

### DETAILED DESCRIPTION OF EMBODIMENTS

The embodiments described and shown herein provide a tubing with a cross-sectional geometry that facilitates reducing the likelihood of kinking. The geometry may provide additional structural support to the tubing such that a kink is less likely to occur and/or that a kink is less likely to completely restrict flow. The geometry may facilitate keeping the tubing flow channel open even if the channel becomes partially compressed, either by kinking or otherwise. The geometry may make the tubing more resilient to kinking by creating tension and/or compression in the walls of the tubing such that kinking is resisted. The tubing cross-sectional geometry may bias the tubing to return to an unkinked state when bent. Further, the cross-section geometry may keep the flow channel within the tubing at least partially open, even if the tubing is partially or totally kinked. In other words, the geometry is configured to bias the tubing against kinking.

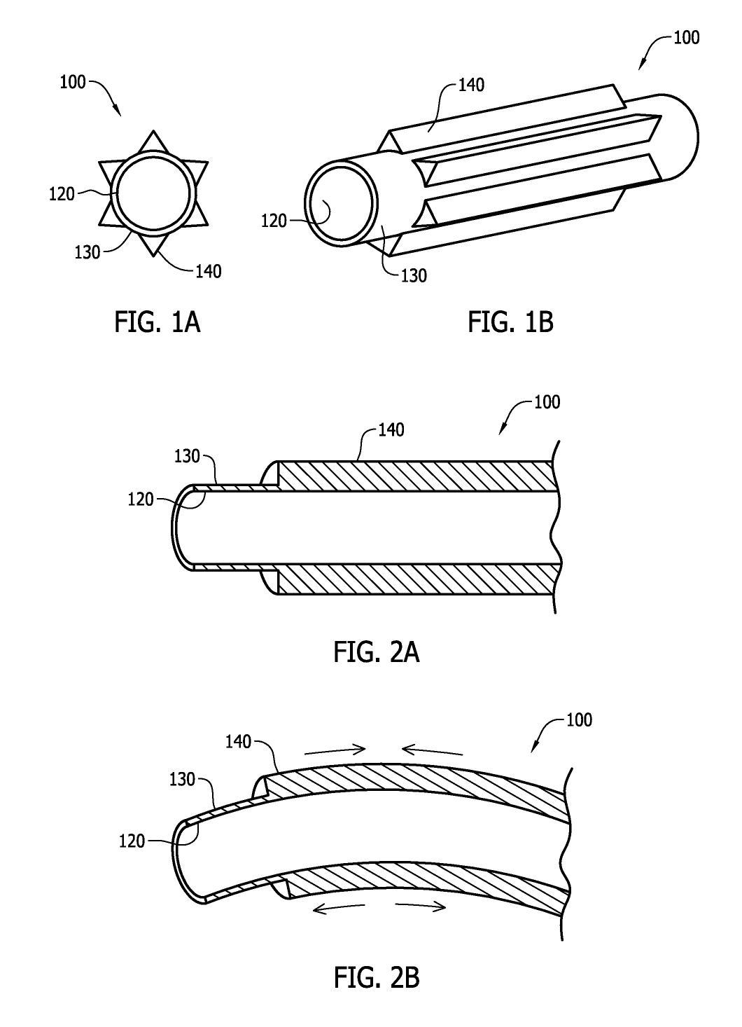

FIG. 1A is an axial view of an anti-kink tubing according to one exemplary embodiment. In this embodiment tubing 100, such as IV tubing, a garden hose, vacuum tubing, or any other tubing or hose that benefits from being configured to resist kinking. In this embodiment, the tubing is formed from a tubular structure defined by an inner surface 120 that defines a tubular cavity through which a fluid (or gas) may flow, an outer surface 130, and a plurality of anti-kinking projections 140. The inner surface 120, outer surface 130, and the anti-kinking projections 140 together make up an anti-kinking cross-sectional geometry. Tubing material exists between the inner surface 120 and the outer surface 130 of the tubing.

Here, each of the anti-kinking projections 140 is a substantially triangular projection that extends from outer surface 130 of the tubing 100. The anti-kinking projections 140 may be formed integrally with the tubing or may be formed separately to be attached to the outer surface 130 of the tubing 100. When the anti-kinking projections 140 are formed separately, they may be attached to the outer surface 130 of the tubing by any suitable method including, but not limited to adhesive boding, fasteners, an interference fit, or the like. For example, the anti-kinking projections 140 may be formed as a part of a sleeve that is placed over the outer surface 130 of the tubing 100.

FIG. 1B is a perspective view of the anti-kink tubing shown in FIG. 1A. As shown in FIG. 1B, the anti-kinking projections 140 extend along the outer surface 130 in an axial direction with respect to the tubing 100. Here, the anti-kinking projections 140 extend along a part of the outer surface 130 and do not extend along the entire length of the tubing 100. In this manner, as shown in FIG. 1B, the anti-kinking features 140 may run parallel to one another along the outer surface 130 of the tubing 100.

The anti-kinking projections 140 may be provided in one or more areas of the tubing 100 that are likely to experience kinking. For example in a garden hose, the anti-kinking projections 140 may be provided near the inlet of the garden hose to prevent kinking near the water source. As another example in IV tubing, the anti-kinking projections 140 may be provided on a more distal part of the IV tubing that is in closer proximity to a patient such that kinking is prevented when the patient moves or rolls over in bed.

The anti-kinking projections 140 need not be provided only to a portion of the tubing 100. The anti-kinking projections 140 may also be provided along an entire length of the tubing 100 to prevent kinking throughout the tubing. The tubing 100 may be further configured so that ends of the tubing 100 may be fitted into couplings to connect the tubing to a fluid inlet and outlet. For example, the tubing 100 may be a garden hose fitted to connect to a water tap. Alternatively, the tubing 100 may be IV tubing that is fitted to connect to a fluid delivery device.

The tubing 100 may be formed of any suitable material for providing sufficient flexibility and strength. The tubing 100 may further be constructed of transparent materials. The properties and amount of materials used for the tubing 100 may be comparable to similar tubing that does not incorporate the anti-kinking geometry as described above.

FIG. 2A is a cross section view of an anti-kink tubing according to an exemplary embodiment, and FIG. 2B is a cross section view of the anti-king tubing shown in FIG. 2A where the anti-kink tubing is curved. The anti-kinking ability of the tubing 100 is explained with reference to FIGS. 2A and 2B. As already discussed, the anti-kinking projections 140 extend along the outer surface 130 of the tubing 100 in an axial direction. In operation, when the tubing 100 is bent from a straight position, as shown in FIG. 2A, to a curved position, as shown in FIG. 2B, one or more anti-kinking projections 140 prevent the tubing from kinking.

Specifically, as shown in FIG. 2B, when the tubing 100 is in a curved state, an anti-kinking projection 140 that is disposed on an inner curvature of the tubing 100 compresses and thus prevents the tubing 100 from kinking. That is, the anti-kinking projection 140 on the inner curvature of the curved tubing 100 applies a force towards the ends of the tubing 100, as indicated by the arrows below the tubing in FIG. 2B. These compression forces in the anti-kinking projection 140 resist excessive curvature in the tubing 100 and therefore prevent kinking in the tubing 100.

Further, as shown in FIG. 2B, when the tubing 100 is in a curved state, an anti-kinking projection 140 that is disposed on an outer curvature of the tubing 100 is placed under tension and thus prevents the tubing 100 from kinking. That is, the anti-kinking projection 140 on the outer curvature of the curved tubing 100 applies a force away from the ends of the tubing 100, as indicated by the arrows above the tubing in FIG. 2B. These tensile forces in the anti-kinking projection 140 resist excessive curvature in the tubing 100 and therefore prevent kinking in the tubing 100.

The above described tubing 100 provides a number of advantages. With the anti-kinking projections 140, the tubing or hose is prevented from becoming kinked. For example with a garden hose, a flow of water may be maintained steadily as the garden hose is pulled to various locations in a user's yard. With IV tubing, at least a distal part of the tubing may incorporate the anti-kinking geometry and prevent kinking when a patient rolls or moves in a bed, ensuring proper delivery of drugs or other fluids. Certain applications require fast fluid flow while other applications utilize flow fluid flow rates. For example, irrigation may require high flow rates while delivery of medicine by an IV line may occur at a slow rate of flow. The design of the various embodiments disclosed herein may be selected based on the flow rate, such as some kinking and reduced flow may be acceptable of certain embodiments, while other embodiments may not allow for any kinking. In addition, the various embodiments may be adopted and used to form any combination.

The anti-kinking geometry is not limited to the above configuration. A number of modification could be made without departing from the scope of the invention, examples of which will be described below.

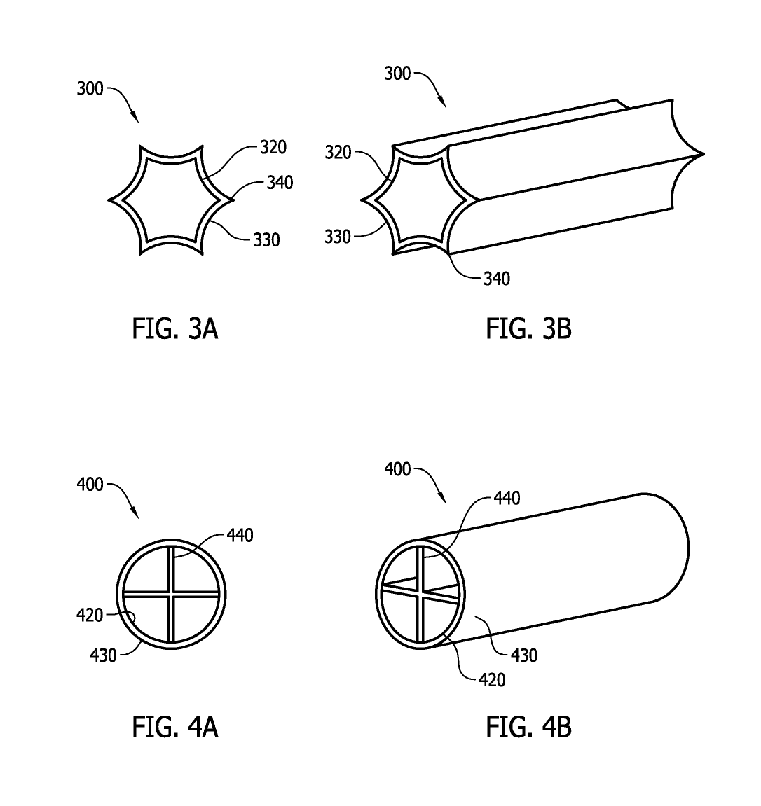

FIG. 3A is an axial view of an anti-kink tubing according to one exemplary embodiment. FIG. 3A shows tubing 300 with another anti-kinking tube geometry that includes an inner surface 320, an outer surface 330, and a plurality of anti-kinking projections 340 formed from the tubing 100.

As shown in FIG. 3A, the anti-kinking projections 340 define a plurality of points projecting away from the outer surface 330. The outer surface 330 is formed into a plurality of concave surfaces that connect the points of the anti-kinking projections 340.

FIG. 3B is a perspective view of the anti-kink tubing shown in FIG. 3A. As shown in FIG. 3B, the outer surface 330 with the plurality of concave surfaces define a plurality of flutes or rounded grooves that extend axially along the length of the tubing 300. Unlike the round inner surface 120 of FIGS. 1A-2B, the tubing 300 has an inner surface 320 with a profile that follows the profile of the outer surface 330.

In this embodiment, less material may be required for the tubing 300 as compared to the tubing 100 because the thickness of the tubing 300 is substantially constant throughout the tubing 300. The tubing 300 provides similar advantages to the tubing 100, as described above, in that anti-kinking projections 340 prevent or inhibit the tubing 300 from kinking.

The tubing 300 shown in FIG. 3A has anti-kinking projections that extend along the entire length of the tubing. However, the tubing 300 may also have the anti-kinking geometry with the anti-kinking projections 340 along only a portion of the length of the tubing 300. This is but one possible embodiment and configuration and it is contemplated that other configurations or shapes are possible.

FIG. 4A is an axial view of an anti-kink tubing according to one exemplary embodiment. FIG. 4A shows tubing 400 with an alternative anti-kinking geometry that includes an inner surface 420, an outer surface 430, and a plurality of anti-kinking projections 440.

As shown in FIG. 4A, the anti-kinking projections 440 are disposed on an inner surface 420 of the tubing 400. Specifically, the anti-kinking projections 440 comprise interior partitions that extend across the interior of the tubing 440 and connect with the interior surface 320. The interior partitions may be formed integrally by joining the partitions in a center thereof, as shown in FIG. 4A. In FIG. 4A, two perpendicular partitions form the anti-kinking projections 440. However, more partitions may also be used and arranged in different configurations, such as equally spaced for uniform kink resistance, or concentrated in certain areas within the inner cavity.

FIG. 4B is a perspective view of the anti-kink tubing shown in FIG. 4A. In FIG. 4B, the anti-kinking projections 440 extend along the entire interior surface 420 of the tubing 400. However, as before, it is possible for the anti-kinking projections 440 to extend only partially along the interior surface 420 of the tubing 400.

The anti-kinking geometry shown in FIGS. 4A and 4B has similar advantages as the other anti-kinking geometries as described above. Further, the anti-kinking geometry in FIGS. 4A and 4B may facilitate the ends of the tubing 400 being connected to standard fittings. Also, because the tubing 400 has a round exterior surface, users of the tubing 400 may be more accustomed to the look and feel of the tubing 400 as compared with the above embodiments.

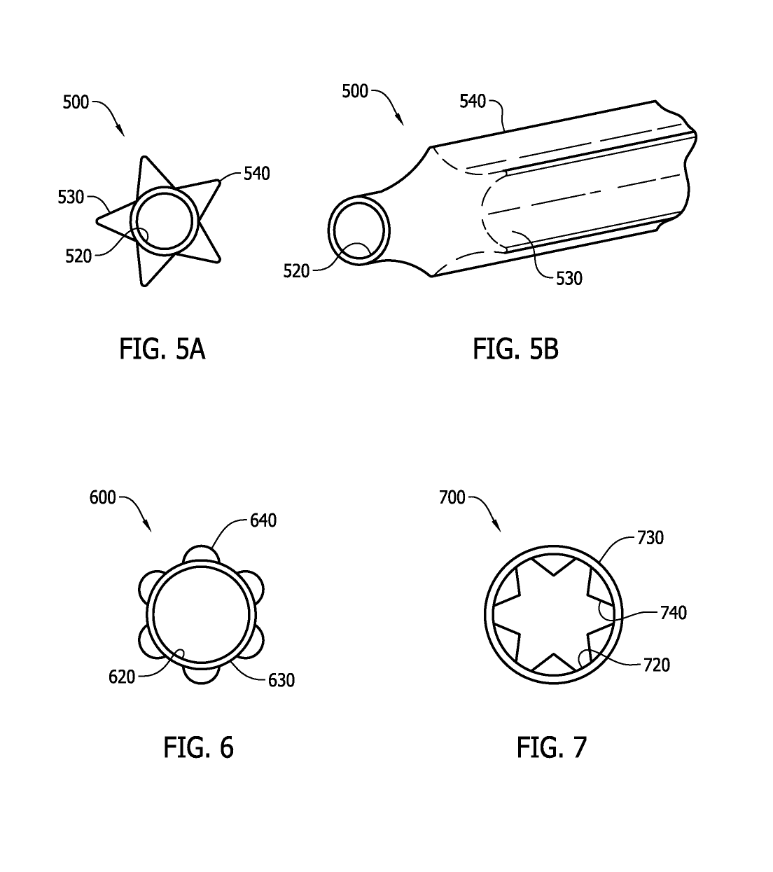

FIG. 5A is an axial view of an anti-kink tubing according to one exemplary embodiment. In the tubing 500 of FIG. 5A, an inner surface 520, outer surface 530, and anti-kinking projections 540 define an anti-kinking geometry. Between the inner surface 520 and the outer surface 530 is the tubing material. In FIG. 5A, the anti-kinking projections are substantially triangular projections extending from the outer surface 530 of the tubing 500.

FIG. 5B is a perspective view of the anti-kink tubing shown in FIG. 5A. FIG. 5B illustrates an exemplary transition between an end of the tubing 500 or a part of the tubing 500 without an anti-kinking geometry to a part of the tubing 500 with an anti-kinking geometry. In FIG. 5, the tubing 500 gradually transitions between a round-shape in cross section at the end, and the anti-kinking geometry in cross section in the mid-section.

In FIGS. 5A and 5B, the tubing 500 is shown where only the outer surface 530 transitions from the round shape to the anti-kinking geometry. However, the inner surface 520 may also transition such that the inner surface 520 profile corresponds with the outer surface 530 profile in the anti-kinking geometry cross section. Or, the inner surface 520 and outer surface may both have anti-kink profiles which are different.

FIG. 6 is an axial view of an anti-kink tubing according to one exemplary embodiment. In the previous embodiments, the anti-kinking projections have been shown as extending to a point. However, the invention is not limited to this. In FIG. 6, tubing 600 includes a further example of anti-kinking geometry. Specifically, tubing 600 has an inner surface 620, an outer surface 630, and anti-kinking projections 640. The anti-kinking projections 640 are formed in a substantially semi-circular, round, or oval shape and extend away from the outer surface 630 of the tubing 600.

In this manner, the anti-kinking projections 640 form reinforcing ribs that extend axially along at least a portion of the length of the outer surface 630 of the tubing 600. In other embodiments, the anti-kinking projections 640 may be of any other suitable shape, such as rectangular, trapezoidal, etc.

FIG. 7 is an axial view of an anti-kink tubing according to one exemplary embodiment. In this embodiment, tubing 700 includes an inner surface 720, an outer surface 730, and a plurality of anti-kinking projections 740. Here, the plurality of anti-kinking projections 740 are disposed on the inner surface 720 of the tubing 700. In FIG. 7, the anti-kinking projections 740 are formed in a substantially triangular shape. However, the anti-kinking projections may be formed in any other suitable shape including semi-circular, rectangular, trapezoidal, etc. The anti-kinking projections 740 operate as reinforcing ribs that extend along at least a portion of the inner surface 720 of the tubing 700.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

For example, while the anti-kinking projections have been described on either the inner surface or the outer surface, anti-kink tubing may comprise anti-kinking projections on both the inner and outer surfaces. The anti-kink tubing further may be tubing that incorporates more than one of the above examples of anti-kink tubing simultaneously.

It is contemplated that any of the embodiments disclosed herein may be configured such that the tube, wall, or projections are made from one or more different materials. For example, the entire structure could be made from one material, or several different materials may be combined to increase the anti-kink properties, durability, flexibility, or reduce weight. In one embodiment, the outer edges or tips of the projections are formed or capped with a material different than the wall of the tube or hose.

It is also contemplated that metal, nylon, or other durable material may be imbedded in the projections or the tube to increase strength or further inhibit kinking of the tube. For example, a wire may run within the projection and generally parallel to the central axis. Wire his highly resistant to compression and stretching and will thus resist kinking. The wire may be coated with a anti-rust coating to reduce corrosion. It is contemplated that not all embodiments will have a wire, such as if weight were a factor.

It is also contemplated that the projections may run from a first end of the hose or tube to a second end of the hose or tube but form a spiral shape around the outer surface or inner surface of the hose or tube. In one embodiment, the angle of the spiral relative to the central axis is 45 degrees or less. This establishes a different configuration than prior art hoses which are wrapped in ridged wire such that the angle of the wire wrap is generally perpendicular to the central axis.

In one or more embodiments, the anti-kink projections may be formed as a sleeve that is configured to slide inside of an existing tube or hose, or slide over an existing hose or tube. In this way, existing hoses may be fit with the anti-kink sleeve to improve an existing hose or tube.

What is claimed is:

1. Anti-kink tubing comprising:
a flexible tube having an inner surface and an outer surface that define an inner cavity extending in an axial direction;
a plurality of anti-kinking projections attached to at least one of the inner and outer surfaces, the plurality of anti-kinking projections extending along the at least one of the inner surface and outer surfaces parallel to the axial direction of the anti-kink tubing.

2. The anti-kink tubing according to claim 1, wherein the anti-kinking projections extend along an entire axial length of the tubing.

3. The anti-kink tubing according to claim 1, wherein the inner surface is substantially circular in cross section, and the anti-kinking projections are formed integrally with the outer surface and protrude from the outer surface.

4. The anti-kink tubing according to claim 1, wherein the anti-kinking projections are formed on the outer surface, and a profile of the inner surface substantially conforms to that of the outer surface.

5. The anti-kink tubing according to claim 1, wherein the anti-kinking projections are formed on the inner surface, and the anti-kinking projections have a first end and a second end that comprise partitions extending radially within an inside of the tubing.

6. The anti-kink tubing according to claim 1, wherein the anti-kinking projections are formed from a different material than the flexible tube.

7. A flexible hose comprising:
A hose defined by a wall having an inner surface and an outer surface which form an inner cavity that extends from a first hose end to a second hose end to define a center axis generally centered in the inner cavity; and
the outer surface forming a plurality of projections extending from the wall to establish the outer surface at different distances from the inner wall based on the circumferential location on the outer surface, the projections forming ribs which extend from the first hose end to the second hose end generally parallel to the central axis.

8. The anti-kink intravenous tubing according to claim 7, further comprising first connector on the first hose end and a second connector on the second hose end.

9. The anti-kink intravenous tubing according to claim 7, wherein the projections are formed in a substantially triangular shape in cross section.

10. The anti-kink intravenous tubing according to claim 7, wherein the projections form a plurality of flutes along the outer surface.

11. The anti-kink intravenous tubing according to claim 7, wherein the projections are formed in a substantially semi-circular shape in cross section.

12. An anti-kink tube comprising
a tube having a central axis, the tube having an inner surface and outer surface which define an inner cavity; and
one or more of anti-kink projections extending from the inner surface within the inner cavity and formed as part of or attached to the inner surface, the anti-kink projections having a extending from the inner surface toward the central axis such that the anti-kink projection run parallel to the central axis of the tube.

13. The anti-kink tube according to claim 12, wherein at least one of the one or more anti-kink projections has a first end and a second end, the second end opposing the first end, such that both the first end and the second end contact the inner wall and the anti-kink projection extends through the central axis.

14. The anti-kink tube according to claim 12, wherein the anti-kink projections are triangle shaped ribs on the inner surfaces.

15. The anti-kink tube according to claim 12, wherein the inner surface and the outer surface are equal distance apart around at least a portion of the circumference of the anti-kink tube.

16. The anti-kink hose according to claim 12, wherein the anti-kink projections comprise two anti-kink projections which are equally spaced within the cavity and each of the two anti-kink projections extend through the central axis.
